(19) United States
(12) Patent Application Publication
(10) Pub. No.: US 2006/0229628 A1
(43) Pub. Date: Oct. 12, 2006

(54) Biomedical treatment systems and methods

(76) Inventors: Csaba Truckai, Saratoga, CA (US); John H. Shadduck, Menlo Park, CA (US)

Correspondence Address: John H. Shadduck, 350 Sharon Park Drive #823, Menlo Park, CA, 94025, US

(21) Appl. No.: 11/242,592

(22) Filed: Oct. 3, 2005

Related U.S. Application Data
(60) Provisional application No. 60/615,559, filed on Oct. 2, 2004.

(51) Int. Cl. A61B 17/58

(52) U.S. Cl. 606/90

(57) ABSTRACT

An apparatus and method for distraction of tissue or bone in a surgery. A method comprises inserting into targeted tissue a distraction device configured with at least one phase transition extendable body. After deployment in a non-extended configuration, the physician applies a stimulus to the body or bodies to cause a liquid-to-vapor or solid-to-vapor phase transition within the body that extends the body in a fast event from the non-extended configuration to an extended configuration to thereby apply distraction forces to the targeted tissue. The extendable body is made of biocompatible materials having any suitable configuration. In one embodiment, the implant and system is used for reducing a vertebral compression fracture. The distraction system can be used to distribute forces over a selected region of strong cortical bone to restore vertebral height. Such a system can be dimensioned as a cylindrical, spherical, annular or part-annular construct for creating selected directional forces for moving apart cortical endplates.

### CROSS-REFERENCE TO RELATED APPLICATIONS

This application claims benefit of Provisional U.S. patent application Ser. No. 60/615,559 filed Oct. 2, 2004 titled Biomedical Implant Systems and Methods of Use. This application also is related to U.S. application Ser. No. 11/165,652 (Atty. Docket No. DFINE.001A1, filed Jun. 24, 2005 titled Bone Treatment Systems and Methods; and U.S. patent application Ser. No. 11/165,651 (Atty. Docket No. DFINE.001A2), filed Jun. 24, 2005, titled Bone Treatment Systems and Methods. The entire contents of all of the above cross-referenced applications are hereby incorporated by reference in their entirety and should be considered a part of this specification.

### BACKGROUND OF THE INVENTION

1. Field of the Invention

The present invention relates to medical devices, and more particularly, to methods and apparatus for applying retraction forces to bone or soft tissue. An exemplary embodiment is used for applying forces to reduce a vertebral fracture. The invention uses phase-change extendable elements that are extendable in response to a stimulus such as temperature to thereby apply expansion forces to a body structure.

2. Description of the Related Art

Osteoporotic fractures are prevalent in the elderly, with an annual estimate of 1.5 million fractures in the United States alone. These include 750,000 vertebral compression fractures (VCFs) and 250,000 hip fractures. The annual cost of osteoporotic fractures in the United States has been estimated at $13.8 billion. The prevalence of VCFs in women age 50 and older has been estimated at 26%. The prevalence increases with age, reaching 40% among 80-year-old women. Medical advances aimed at slowing or arresting bone loss from aging have not provided solutions to this problem. Further, the affected population will grow steadily as life expectancy increases. Osteoporosis affects the entire skeleton but most commonly causes fractures in the spine and hip. Spinal or vertebral fractures also have serious consequences, with patients suffering from loss of height, deformity and persistent pain which can significantly impair mobility and quality of life. Fracture pain usually lasts 4 to 6 weeks, with intense pain at the fracture site. Chronic pain often occurs when one level is greatly collapsed or multiple levels are collapsed.

Postmenopausal women are predisposed to fractures, such as in the vertebrae, due to a decrease in bone mineral density that accompanies postmenopausal osteoporosis. Osteoporosis is a pathologic state that literally means “porous bones”. Skeletal bones are made up of a thick cortical shell and a strong inner meshwork, or cancellous bone, of collagen, calcium salts and other minerals. Cancellous bone is similar to a honeycomb, with blood vessels and bone marrow in the spaces. Osteoporosis describes a condition of decreased bone mass that leads to fragile bones which are at an increased risk for fractures. In an osteoporotic bone, the sponge-like cancellous bone has pores or voids that increase in dimension, making the bone very fragile. In young, healthy bone tissue, bone breakdown occurs continually as the result of osteoclast activity, but the breakdown is balanced by new bone formation by osteoblasts. In an elderly patient, bone resorption can surpass bone formation thus resulting in deterioration of bone density. Osteoporosis occurs largely without symptoms until a fracture occurs.

Vertebroplasty and kyphoplasty are recently developed techniques for treating vertebral compression fractures. Percutaneous vertebroplasty was first reported by a French group in 1987 for the treatment of painful hemangiomas. In the 1990's, percutaneous vertebroplasty was extended to indications including osteoporotic vertebral compression fractures, traumatic compression fractures, and painful vertebral metastasis. In one percutaneous vertebroplasty technique, bone cement such as PMMA (polymethylmethacrylate) is percutaneously injected into a fractured vertebral body via a trocar and cannula system. The targeted vertebrae are identified under fluoroscopy. A needle is introduced into the vertebral body under fluoroscopic control to allow direct visualization. A transpedicular (through the pedicle of the vertebrae) approach is typically bilateral but can be done unilaterally. The bilateral transpedicular approach is typically used because inadequate PMMA infill is achieved with a unilateral approach.

In a bilateral approach, approximately 1 to 4 ml of PMMA are injected on each side of the vertebra. Since the PMMA needs to be forced into cancellous bone, the technique requires high pressures and fairly low viscosity cement. Since the cortical bone of the targeted vertebra may have a recent fracture, there is the potential of PMMA leakage. The PMMA cement contains radiopaque materials so that when injected under live fluoroscopy, cement localization and leakage can be observed. The visualization of PMMA injection and extravasion are critical to the technique—and the physician terminates PMMA injection when leakage is evident. The cement is injected using small syringe-like injectors to allow the physician to manually control the injection pressures.

Kyphoplasty is a modification of percutaneous vertebroplasty. Kyphoplasty involves a preliminary step that comprises the percutaneous placement of an inflatable balloon tamp in the vertebral body. Inflation of the balloon creates a cavity in the bone prior to cement injection. Further, the proponents of percutaneous kyphoplasty have suggested that high pressure balloon-tamp inflation can at least partially restore vertebral body height. In kyphoplasty, it has been proposed that PMMA can be injected at lower pressures into the collapsed vertebra since a cavity exists to receive the cement—which is not the case in conventional vertebroplasty.

The principal indications for any form of vertebroplasty are osteoporotic vertebral collapse with debilitating pain. Radiography and computed tomography must be performed in the days preceding treatment to determine the extent of vertebral collapse, the presence of epidural or foraminal stenosis caused by bone fragment retropulsion, the presence of cortical destruction or fracture and the visibility and degree of involvement of the pedicles. Leakage of PMMA during vertebroplasty can result in very serious complications including compression of adjacent structures that necessitate emergency decompressive surgery.

Leakage or extravasion of PMMA is a critical issue and can be divided into paravertebral leakage, venous infiltration, epidural leakage and intradiscal leakage. The exothermic reaction of PMMA carries potential catastrophic consequences if thermal damage were to extend to the dural sac, cord, and nerve roots. Surgical evacuation of leaked cement in the spinal canal has been reported. It has been found that leakage of PMMA is related to various clinical factors such as the vertebral compression pattern, and the extent of the cortical fracture, bone mineral density, the interval from injury to operation, the amount of PMMA injected and the location of the injector tip. In one recent study, close to 50% of vertebroplasty cases resulted in leakage of PMMA from the vertebral bodies. See Hyun-Woo Do et al, “The Analysis of Polymethylmethacrylate Leakage after Vertebroplasty for Vertebral Body Compression Fractures”, Jour. of Korean Neurosurg. Soc. Vol. 35, No. 5 (5/2004) pp. 478-82, (http://wwwjkns.or.kr/htm/abstract.asp?no=0042004086).

Another recent study was directed to the incidence of new VCFs adjacent to the vertebral bodies that were initially treated. Vertebroplasty patients often return with new pain caused by a new vertebral body fracture. Leakage of cement into an adjacent disc space during vertebroplasty increases the risk of a new fracture of adjacent vertebral bodies. See Am. J. Neuroradiol. 2004 Feb; 25(2): 175-80. The study found that 58% of vertebral bodies adjacent to a disc with cement leakage fractured during the follow-up period compared with 12% of vertebral bodies adjacent to a disc without cement leakage.

Another life-threatening complication of vertebroplasty is pulmonary embolism. See Bernhard, J. et al., “Asymptomatic diffuse pulmonary embolism caused by acrylic cement: an unusual complication of percutaneous vertebroplasty”, Ann. Rheum. Dis. 2003; 62:85-86. The vapors from PMMA preparation and injection are also cause for concern. See Kirby, B., et al., “Acute bronchospasm due to exposure to polymethylmethacrylate vapors during percutaneous vertebroplasty”, Am. J. Roentgenol. 2003; 180:543-544.

Another disadvantage of PMMA is its inability to undergo remodeling—and the inability to use the PMMA to deliver osteoinductive agents, growth factors, chemotherapeutic agents and the like. Yet another disadvantage of PMMA is the need to add radiopaque agents which lower its viscosity with unclear consequences on its long-term endurance.

In both higher pressure cement injection (vertebroplasty) and balloon-tamped cementing procedures (kyphoplasty), the methods do not provide for well controlled augmentation of vertebral body height. The direct injection of bone cement simply follows the path of least resistance within the fractured bone. The expansion of a balloon also applies compacting forces along lines of least resistance in the collapsed cancellous bone. Thus, the reduction of a vertebral compression fracture is not optimized or controlled in high pressure balloons as forces of balloon expansion occur in multiple directions.

In a kyphoplasty procedure, the physician often uses very high pressures (e.g., up to 200 or 300 psi) to inflate the balloon which first crushes and compacts cancellous bone. Expansion of the balloon under high pressures close to cortical bone can fracture the cortical bone, or cause regional damage to the cortical bone that can result in cortical bone necrosis. Such cortical bone damage is highly undesirable and results in weakened cortical endplates.

Kyphoplasty also does not provide a distraction mechanism capable of 100% vertebral height restoration. Further, the kyphoplasty balloons under very high pressure typically apply forces to vertebral endplates within a central region of the cortical bone that may be weak, rather than distributing forces over the endplate.

There is a general need to provide systems and methods for use in treatment of vertebral compression fractures that provide a greater degree of control over introduction of bone support material, and that provide better outcomes. Embodiments of the present invention meet one or more of the above needs, or other needs, and provide several other advantages in a novel and non-obvious manner.

### SUMMARY OF THE INVENTION

The invention provides implant systems and methods for treatment of vertebral compression fractures, as well as systems for prophylactic treatment of osteoporotic vertebrae in patients that are susceptible to compression fractures. The invention also can be used in correcting and supporting bones in other abnormalities such as bone tumors and cysts, avascular necrosis of the femoral head and tibial plateau fractures. The invention also can be used for tissue distraction or retraction in any other soft tissues or mammalian body structures.

In general, a preferred method of the invention relates to distraction of tissue in a surgery, and comprises the steps of inserting into targeted tissue a distraction device configured with at least one phase transition extendable body—with said extendable body in a non-extended configuration. Thereafter, a stimulus is applied to the body or bodies to cause a liquid-to-vapor or solid-to-vapor phase transition within the body that extends the at least one extendable body in a fast event from the non-extended configuration to an extended configuration to thereby apply distraction forces to the targeted tissue. In one embodiment, the extendable body is made of biocompatible materials having any suitable configuration and can be used to distribute forces over a selected region of cortical bone to reduce a compression fracture. Such a system can be dimensioned as a spherical, annular or part-annular construct for creating selected directional forces for jacking apart cortical endplates to augment vertebral body height. While the structure maintains the restored vertebral height, a flowable polymer such as PMMA optionally can be introduced under pressure into region around the structure to intercalate and harden within the cancellous bone.

In one embodiment, the invention provides an implant system that allows from controlled forces for moving cortical bone in a collapsed vertebra. The invention provides a system that allows for the reduction or elimination of exothermic effects of bone cement that may be undesirable.

These and other objects of the present invention will become readily apparent upon further review of the following drawings and specification.

### BRIEF DESCRIPTION OF THE DRAWINGS

In order to better understand the invention and to see how it may be carried out in practice, some preferred embodiments are next described, by way of non-limiting examples only, with reference to the accompanying drawings, in which like reference characters denote corresponding features consistently throughout similar embodiments in the attached drawings.

FIG. 1 is a schematic representation of an osteoporotic vertebral body having a wedge compression fracture within phantom view of its original pre-fracture height, further indicating the location of an implant structure of the invention being introduced in the vertebra.

FIG. 2A is a sectional view of a vertebra indicating an optional location and configuration of an implant structure.

FIG. 2A is a sectional view of a vertebra indicating first and second locations of implant structures.

FIG. 3 is a representation similar to FIG. 1 illustrating an objective of the invention in restoring vertebral height; further indicating a specialized configuration of implant adapted for vertebral jacking forces.

FIG. 4 is a sectional view of a vertebra with illustrating the step of introducing the implant of FIG. 3.

FIG. 5A is a perspective view of the implant of FIG. 3 in a first shape.

FIG. 5B is a perspective view of the implant of FIGS. 3 and 5A in a second extended shape.

FIG. 6A is a side view of a phase change extendable element that is used in the implant body of FIGS. 5A and 5B in a non-extended configuration.

FIG. 6B is a sectional view of a phase change extendable element of FIG. 6A.

FIG. 7A is a side view of a phase change extendable element of FIGS. 6A and 6B in an extended configuration.

FIG. 7B is a sectional view of a phase change extendable element of FIG. 7A.

FIG. 8A is a side view of an alternative burstable phase change extendable element similar to that FIGS. 6A and 7A in a non-extended configuration.

FIG. 8B is a sectional view of the phase change extendable element of FIG. 8A.

### DETAILED DESCRIPTION OF THE INVENTION

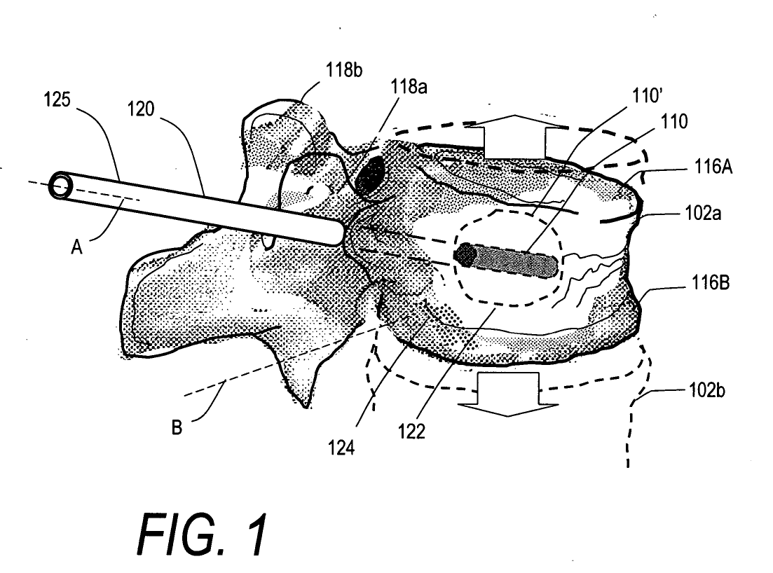

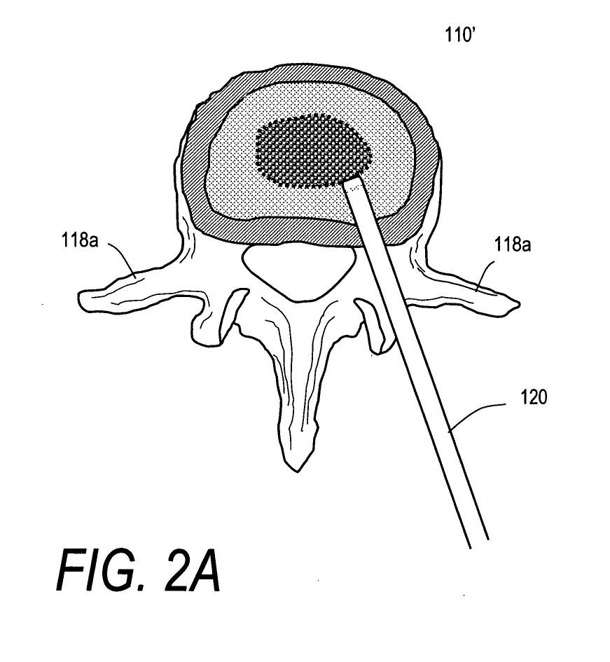

In FIG. 1, it can be seen that vertebral body 102a has a “wedge” compression fracture indicated at 104 and the method of the invention is directed to elevating the vertebral body height while preserving cancellous bone for reasons described below. The implant 110 comprises a self-contained structure that can be altered in-situ from a first reduced cross-sectional configuration (110) to a second extended or expanded cross-sectional configuration (110′) to apply retraction forces to the vertebral body. The device and method include using a plurality of fast-event expansive or explosively expansive elements or bodies 115 that are either (i) introduced loosely into the targeted tissue, or (ii) carried in a confining structure that can be a mesh, knit, woven, braided, perforated, resilient, elastic or inelastic material for generally confining the elements in a selected region. The implant structure 110 can atraumatically engage and apply distraction forces to cortical endplates 116A and 116B.

More in particular, FIG. 1 illustrates an initial step wherein the working end 120 of an elongate instrument is introduced to the saddle of pedicle 118a for penetration therethrough along axis A into the osteoporotic cancellous bone 122. It should be appreciated that the instrument also can be introduced at any other location, for example, through the wall 124 of the vertebral body as indicated along axis B in FIG. 1. In FIG. 1, any cutting or other penetrating tip known in the art may be used to create access through the pedicle.

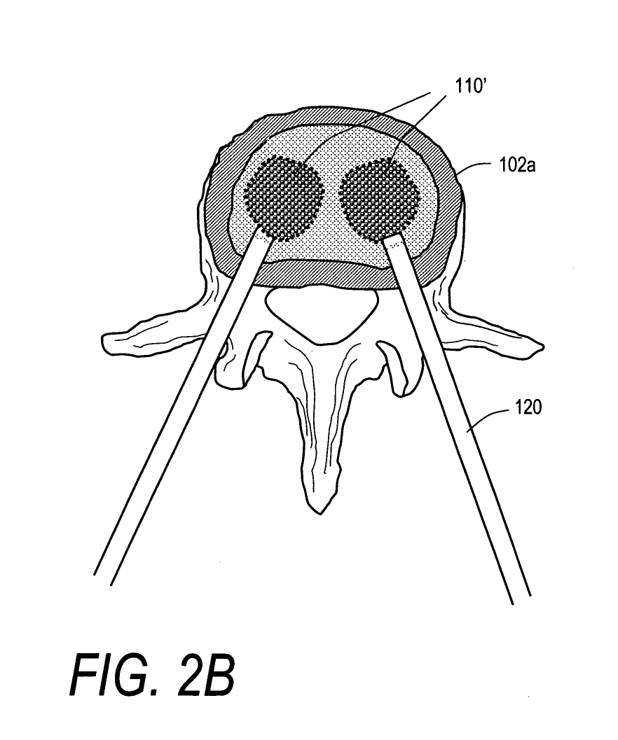

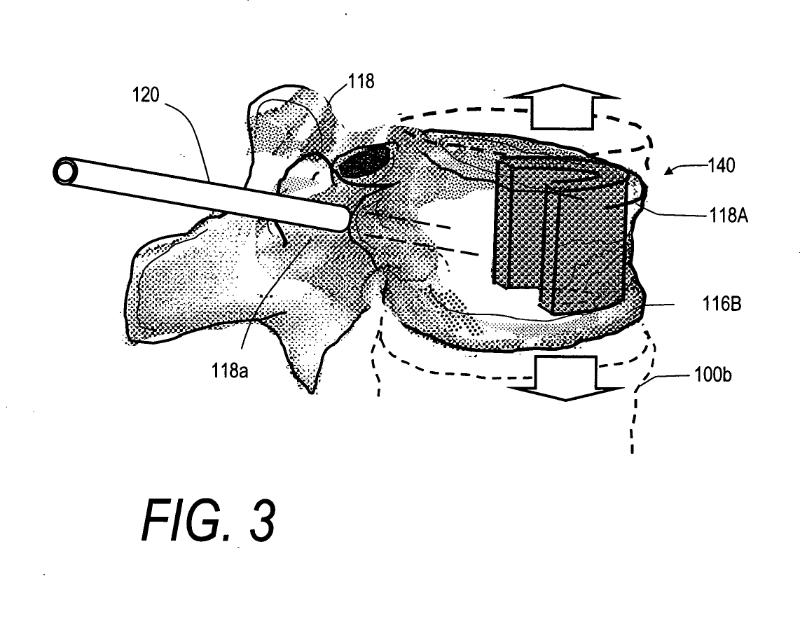

FIG. 3 illustrates another implant structure 140 that has a specialized configuration for preserving the bulk of cancellous bone in the central portion of the vertebral body and then is adapted to apply strong distraction forces to the vertebral endplates about an anterior portion to reduce the vertebral compression fracture. FIG. 4 shows the shape of the implant structure 140 as it is introduced into a path made by cutting, drilling, grinding or simply pushing an instrument working end distally into the bone.

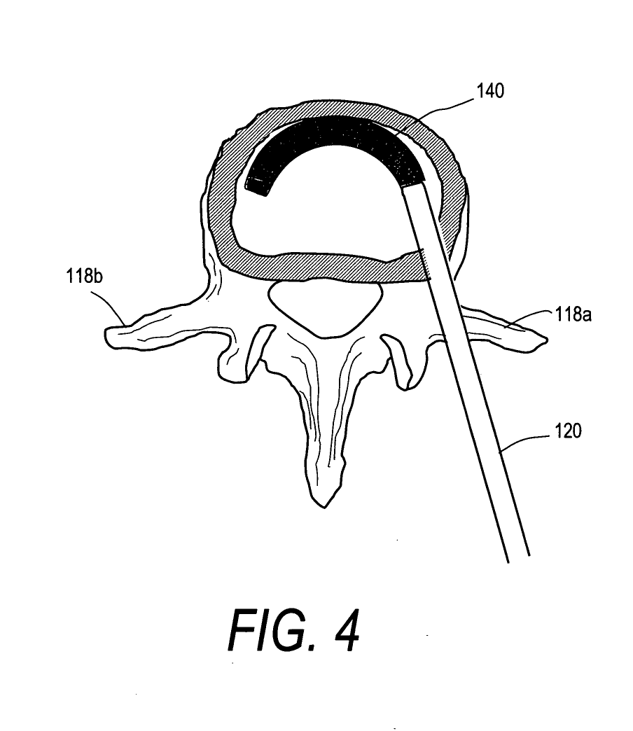

FIG. 5A illustrates the implant structure 140 in a reduced cross-sectional configuration. In FIG. 5B, the implant structure indicated at 140′ is altered to an extended cross-sectional configuration. In the interior of the structure, a plurality of phase change extendable elements 150 can be extended to extended configurations (150′). In FIGS. 5A and 5B, the phase change extendable elements or bodies 150 can be carried in a plurality or tubular, woven or braided, axially-collapsed elements 155. Upon the extension of elements 150, the structure 140 is constrained to extend in selected directions as indicated by the arrows in FIG. 5B. The overall material 156 making up structure 140 that carries and maintains the orientation of the multiple tubular, woven or braided elements 155 can itself be at least one of knit, woven, braided or it can be collapsible polymer foam.

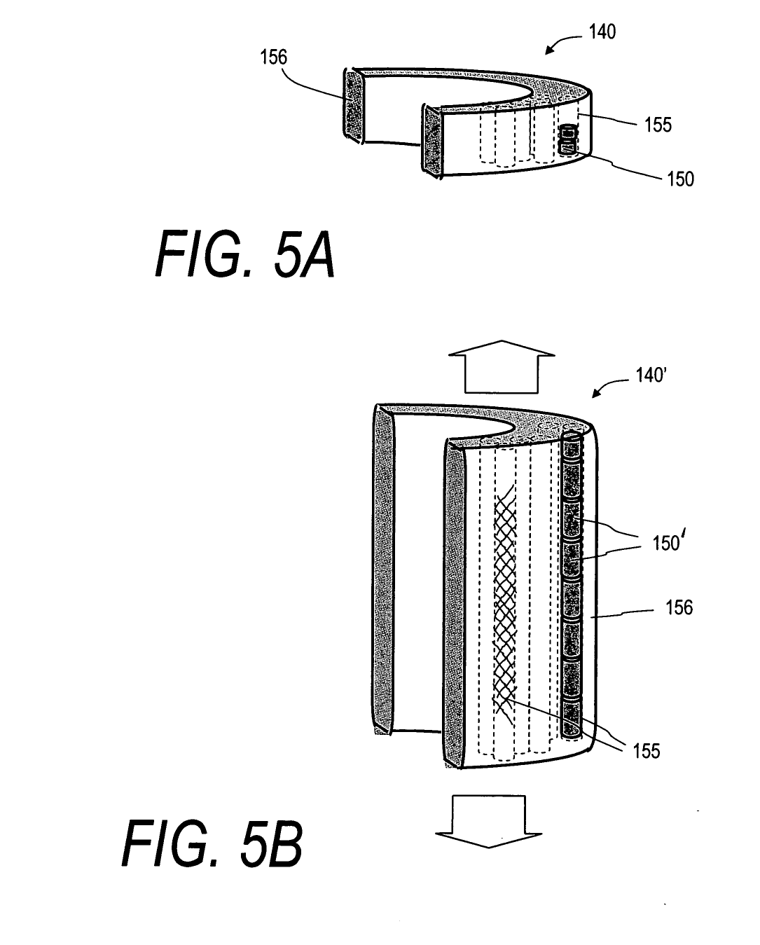

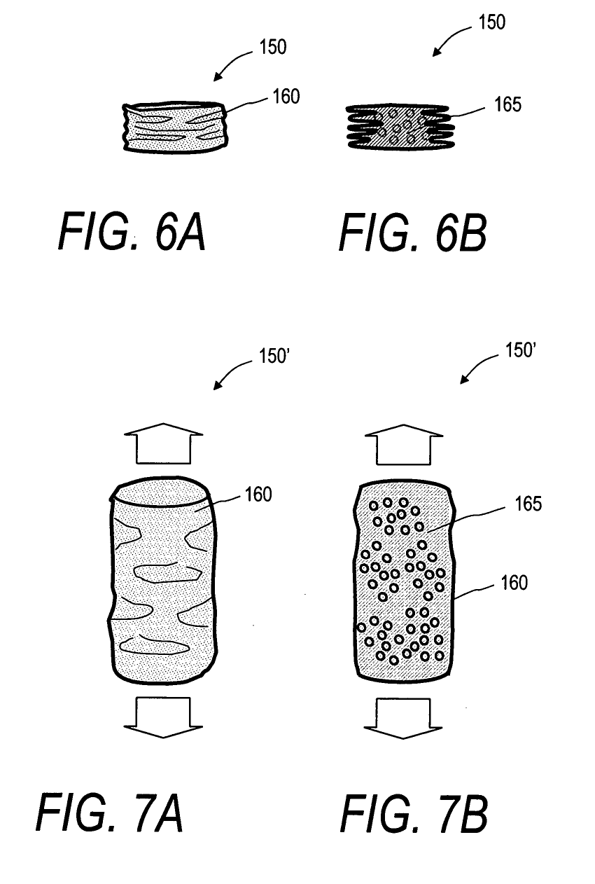

FIGS. 6A and 6B illustrate plan and sectional views of a single phase change extendable element or body 150 in its pre-deployed, non-extended configuration. In FIG. 6B, it can be seen that the element comprises a structural wall 160 that surrounds an interior phase-transitionable media 165. FIGS. 7A and 7B illustrate the corresponding plan and sectional views of the extendable element 150′ in its deployed and extended configuration. In one embodiment, the element in a collapsed form can be any dimension from about 25 microns along a principal axis to over 5.0 mm along the principal axis. Preferably, the element is from about 250 microns to 2.0 mm along the principal axis thereof. The structural wall 160 of the element 150 is typically a metal for providing structure strength but the wall or shell 160 also can be a polymer. The structural wall 160 is preferably substantially fluid impermeable, but also can be microporous. In one embodiment, the interior phase-transitionable media 165 is water, saline solution, a hydrogel or another superabsorbent polymer (superporous hydrogel) that retains water. A hydrogel is a three-dimensional network of hydrophilic polymer chains that are crosslinked through either chemical or physical bonding. Because of the hydrophilic nature of polymer chains, hydrogels absorb water to swell in the presence of abundant water. The swelling process is the same as the dissolution of non-crosslinked hydrophilic polymers. By definition, water constitutes at least 10% of the total weight (or volume) of a hydrogel. When the content of water exceeds 95% of the total weight (or volume), the hydrogel is called a superabsorbent. In a chemical hydrogel, all polymer chains are crosslinked to each other by covalent bonds, and thus, the hydrogel is one molecule regardless of its size. For this reason, there is no concept of molecular weight of hydrogels, and hydrogels are sometimes called infinitely large molecules or supermacromolecules. One of the unique properties of hydrogels is their ability to maintain original shape during and after swelling due to isotropic swelling. Dried hydrogels, also called xerogels, can used in fabricating the elements 150, and are particularly appropriate when cut into small particles to allow fast swelling (i.e., swelling in a matter of minutes rather than hours). Fast swelling with very small particles of dried hydrogels is possible due to the extremely short diffusion path lengths of microparticles. Larger dried hydrogels also can be used and made to swell in a matter of minutes by making porous interconnections throughout the hydrogel matrix. Such interconnected pores allow for fast absorption of water by capillary force, and the production of gas bubbles during crosslinking of the polymer can be used to make such a superporous hydrogel or foam. Superporous hydrogels can be synthesized in a mold to allow a three-dimensional structure of any shape.

An exemplary method of fabricating an extendable element or body 150 is as follows. Fabricate and form a macroporous hydrogel or other hydrogel having interconnected pores in the 100 nm to 1 mm range into a polymer body, dry the body, and create a micro- or nanoporous coating of at least one of a metal, polymer or ceramic. This process would create a body as in FIGS. 5A-5B, with a preferred embodiment having a convoluted, folded or bellow-like wall structure. Thereafter, a plurality of elements are soaked in water for a suitable period to allow the hydrogel to uptake a maximum amount of fluid. The structural shell layer has a required strength to prevent expansion of the polymer. Optionally thereafter, a sealing layer may be applied to the elements. A selected quantity of such elements then can be loaded into a structure 140 as in FIG. 5A.

In one preferred embodiment, a system of electroless plating is used to create the structural wall 160. The wall can be any biocompatible metal with electroless plating used to create any suitable thickness and strength. In this embodiment, the polymer will add strength to the body 150′ in its extended shape as in FIGS. 7A-7B. It should be appreciated that the elements also can be a metal shell with water or saline solution therein—without a polymer component in the interior of the body. In this case, the deformable metal wall would provide all the element's strength in the expanded position.

In the embodiment of FIGS. 5A-5B, the structure 140 extends in a part-annular shape having a radius ranging between about 10 mm. and 50 mm. The thickness can be any suitable dimension.

In operation, any energy source can be use to elevate the temperature of the water or other fluid in the interior media 165 to undergo a phase transition to thereby explosively expand to extend each element to its extended configuration. Water undergoes an expansion of up to 1700 times it original liquid volume in a liquid to vapor transition so it can be understood that very high expansion pressures can be created.

In one embodiment operation, the metal shells 160 can be heated resistively or heated by a laser or other light energy source as is known in the art. Alternatively, Rf or microwave energy can be used to vaporize saline or water in the elements. Another alternative is to use ultrasound energy to heat the phase-extendable bodies. Another alternative is to use inductive heating to heat the phase-extendable bodies.

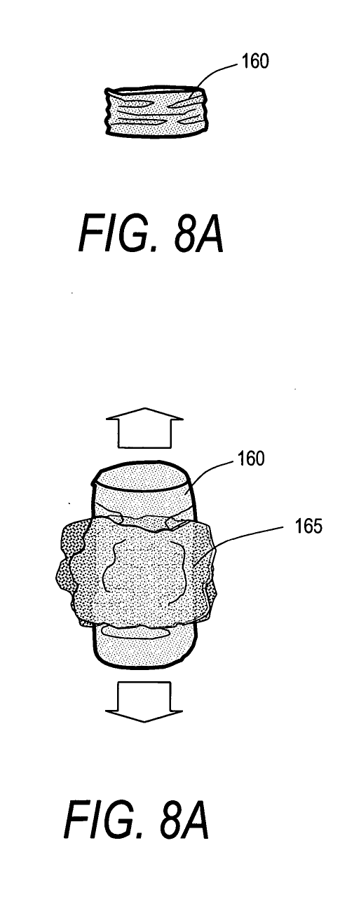

FIGS. 8A and 8B show another embodiment wherein the shell designed for fracturing and the polymer portion then is adapted to carry loads.

In another embodiment (not shown), a structural shell 160 similar to that of FIG. 5A carries a liquefied gas at a suitable low temperature, such as liquid CO₂, nitrogen, or oxygen. The elements are kept cool before deployment. Following deployment, body temperature then causes the phase change in the media to cause extension of the bodies for tissue distraction purposes.

In another embodiment (not shown), the extendable body is any naturally occurring seed, grain or the like that has a vitreous-like or starch shell and that carries water in its interior that can be vaporized. Such seeds as popcorn and amaranth seeds are known to be poppable, and it is believed would be biocompatible. The scope of the invention extends to fabrication of synthetic “seeds” having a starch interior and a vitreous starch shell.

In another embodiment, the wall material 160 can include scaffold elements that carry at least in part a polymeric material configured for timed release of a pharmacological or bioactive agent (e.g., any form of BMP, an antibiotic, an agent that promotes angiogenesis, etc.). Smaller scaffold elements can have a mean pore cross section ranging from 5 nanometers to 100 microns. Larger scaffold elements can have mean pore cross sections ranging from 100 microns to 2000 microns. In one embodiment, the scaffold element are fabricated by e-spinning methods disclosed in co-pending Provisional U.S. patent application Ser. No. 60/588,728 filed Jul. 16, 2004 titled Orthopedic Scaffold Constructs, Methods of Use and Methods of Fabrication, which is incorporated herein in its entirety by this reference.

In another embodiment, the body 156 can comprise a polymeric open cell construct that carries insulative microspheres in the webs of the open cells which can substantially reduce conductive heat transfer from any phase change heat in the bodies 150. Only the level of heat transfer desired is released by control of the volume of insulative microspheres of glass, ceramic or polymers. Such insulative microspheres are available from Potters Industries Inc., P.O. Box 840, Valley Forge, Pa. 19482, for example, microspheres marketed under the names of Spheriglass®, Sphericel® and Q-Cel®.

The scope of the invention includes any working ends for any surgical tissue or bone distraction procedure that carries phase expandable structures in any shape or configuration. In any embodiment, the annual implant structure can include additional radiopaque materials.

In any method of use, the implant or surrounding region in a vertebra also can be infilled with a PMMA or other bone cement following use of the implant to reduce a vertebral fracture.

The above description of the invention intended to be illustrative and not exhaustive. A number of variations and alternatives will be apparent to one having ordinary skills in the art. Such alternatives and variations are intended to be included within the scope of the claims. Particular features that are presented in dependent claims can be combined and fall within the scope of the invention. The invention also encompasses embodiments as if dependent claims were alternatively written in a multiple dependent claim format with reference to other independent claims.

1.-28. (canceled)

29. A method for distraction of mammalian body structure, comprising the steps of:
(a) inserting into a mammalian body at least one phase transition extendable body, said extendable body in a non-extended configuration; and
(b) providing a stimulus thereby causing a liquid-to-vapor or solid-to-vapor phase transition of media within the at least one extendable body to move the extendable body from a non-extended configuration to an extended configuration thereby applying distraction forces on the body structure.

30. The method of claim 29 wherein step (b) applies distraction forces to at least one of soft tissue and bone.

31. The method of claim 29 wherein step (b) applies distraction forces to a bone selected from the class consisting of a vertebra, femur or tibia.

32. The method of claim 29 wherein step (b) applies distraction forces to cortical bone.

33. The method of claim 29 wherein step (b) applies distraction forces to cancellous bone.

34. The method of claim 29 wherein step (b) applies distraction forces within a vertebral body to reduce a fracture.

35. The method of claim 29 wherein the stimulus is temperature.

36. The method of claim 29 wherein the media has a phase transition temperature between about 30° C. and 150° C.

37. The method of claim 29 wherein the media is selected from the group consisting of water, saline solution, nitrogen and carbon dioxide.

38. The method of claim 29 wherein the stimulus is provided by a source selected from the group of an Rf source, a resistive heat source, a light source, an ultrasound source, a microwave source and body temperature.

39. A surgical distraction system comprising at least one phase extendable body that is alterable from a non-extended configuration to an extended configuration to thereby apply distraction forces to tissue.

40. The surgical distraction system of claim 39 wherein each phase extendable body includes a phase transitionable media responsive to a selected stimulus to cause a liquid-to-vapor or solid-to-vapor phase transition therein.

41. The surgical distraction system of claim 39 wherein the structure is selected from the class consisting of distensible wall structures, non-distensible wall structures, braided structures, woven structures, knit structures and mesh structures.

42. The surgical distraction system of claim 39 wherein each phase extendable body defines an axis about which the body substantially extends from the non-extended configuration to the extended configuration.

43. The surgical distraction system of claim 39 wherein each phase extendable body includes a substantially fluid impermeable shell.

44. The surgical distraction system of claim 43 wherein said fluid impermeable shell is at least one of deformable and explodable.

45. The surgical distraction system of claim 43 wherein said fluid impermeable shell is at least one of a metal, plastic, glass and ceramic.

46. The surgical distraction system of claim 39 further comprising a cooling source for maintaining the phase extendable bodies in a non-extended configuration.

47. The surgical distraction system of claim 39 further comprising a heating source for altering the phase extendable bodies from the non-extended configuration to the extended configuration.

48. The surgical distraction system of claim 47 wherein the heating source is selected from the class consisting of Rf sources, resistive heating sources, laser sources ultrasound sources and microwave sources.
